(19) United States
(12) Patent Application Publication
(10) Pub. No.: US 2005/0232983 A1
(43) Pub. Date: Oct. 20, 2005

(54) Transdermal patch

(75) Inventor: Bobby W. Sandage JR., Acton, MA (US)

Correspondence Address: KATTEN MUCHIN ROSENMAN LLP, 525 WEST MONROE STREET, CHICAGO, IL, 60661-3693, US

(73) Assignee: INDEVUS PHARMACEUTICALS, INC., Lexington, MA (US)

(21) Appl. No.: 11/103,504

(22) Filed: Apr. 12, 2005

Related U.S. Application Data
(60) Provisional application No. 60/561,900, filed on Apr. 14, 2004.

(51) Int. Cl. A61K 9/70; A61K 31/4745

(52) U.S. Cl. 424/449; 514/300

(57) ABSTRACT

A transdermal delivery system for pyrrole derivatives of formula 1, metabolites thereof, enantiomers thereof, racemates thereof, and salts including acid addition salts thereof, alone or in conjunction with one or more other therapeutic agents, using vehicles selected to enhance absorption and which enable the compounds to be administered into and through the skin when topically applied.

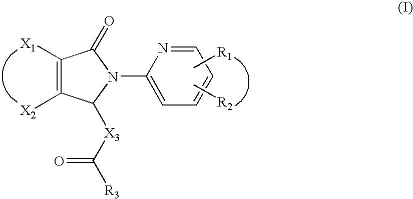

### BACKGROUND OF THE INVENTION

1. Field of the Invention

Transdermal administration of cyclopyrrolones of formula 1.

2. Discussion of the Background

The products of general formula 1 have anxiolytic, hypnotic, anticonvulsant, antiepileptic and muscle relaxant activity They are described in U.S. Pat. No. 4,960,779; incorporated by reference in its entirety.

Transdermal delivery of the subject compounds provides a non-invasive method for the controlled release and delivery of the active agent. These drugs have been difficult to administer transdermally, due to the low skin flux or permeation rates of the drug and the amount of drug that must be delivered for therapeutic efficacy. The present invention discloses vehicles which enhance the rate of drug absorption through the skin.

Transdermal administration is advantageous compared to conventional dosing methods that may result in underdosing or overdosing of the drug. Other advantages include reduction in dosing frequency, reduced fluctuation in circulating drug levels, increased patient compliance and convenience, and a more uniform effect.

In general, the patch of the invention uses a pressure-sensitive medical grade silicone adhesive material which contains the active compound together with and a permeation enhancer. A “permeation enhancer” is defined as a compound compatible with compounds of formula 1 that facilitates its uptake through the skin and thus enables a therapeutically effective dosage to be administered to the patient. One class of permeation enhancers contemplated are aromatic or aliphatic carbocyclic compounds that have pendant hydroxyl groups, such as butylated hydroxytoluene (BHT), butylated hydroxyanisole (BHA), a hydroxypropyl-beta-cyclodextrin (HPBCD), and the like, as well as mixtures thereof.

In addition, lecithin enhances the penetration of compounds of formula 1 through the skin.

### SUMMARY OF THE INVENTION

One object of the invention is to provide A transdermal patch for topical administration of a cyclopyrrolone compound, comprising:
a) a backing layer;
b) a drug depot selected from the group consisting of a liquid reservoir and a monolithic matrix; said depot comprising a compound of formula 1, and a permeation enhancer composition; and
c) means for affixing the patch to the skin of the patient; formula 1: 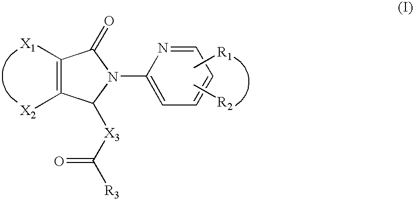
   as defined below.

Another object is to provide a transdermal patch suitable for a once-a-week administration of pagoclone, an active metabolite thereof, or prodrugs of the foregoing comprising an amount of pagoclone, an active metabolite thereof, or prodrugs of the foregoing contained in a transdermal delivery system for the controlled release of pagoclone, an active metabolite thereof, or prodrugs of the foregoing, such that an average plasma level of pagoclone, an active metabolite thereof, or prodrugs of the foregoing falling in the range of about 0.05 ng/ml to about 25 ng/ml is achieved over a one-week period.

Another object is to provide a transdermal patch suitable for a once-a-week administration of pagoclone, an active metabolite thereof, or prodrugs of the foregoing comprising an amount of pagoclone, an active metabolite thereof, or prodrugs of the foregoing contained in a transdermal delivery system for the controlled release of pagoclone, an active metabolite thereof, or prodrugs of the foregoing, such that once-a-week application of said transdermal patch provides steady state blood levels of pagoclone, an active metabolite thereof, or prodrugs of the foregoing, which are substantially bioequivalent to steady state blood levels of pagoclone, an active metabolite thereof, or prodrugs of the foregoing achieved with once daily oral administration of pagoclone, an active metabolite thereof, or prodrugs of the foregoing at an oral daily dosage level falling in the range of about 0.1 mg to about 1 mg.

### DESCRIPTION OF PREFERRED EMBODIMENTS

Compounds of the invention are cyclopyrrolones according to Formula 1, metabolites thereof, enantiomers thereof, racemates thereof, and salts including acid addition salts thereof, alone or in conjunction with one or more other therapeutic agents: 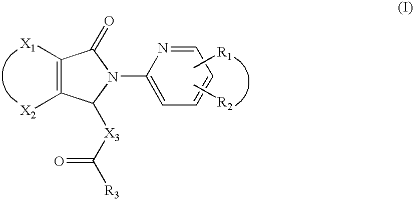
wherein:
(a) R₁ and R₂ are the same or different sterically compatible substituents which are selected from the group consisting o£ hydrogen; alkyl having 1 to 8 carbon atoms; alkyl having 1 to 8 carbon atoms, and having at least one of nitrogen, oxygen, sulfur, or phosphorus; aryl having 1 to 8 carbon atoms; and aryl having 1 to 8 carbon atoms and having at least one nitrogen, oxygen, sulfur, or phosphorus;
(b) R₃ is selected from the group of substituents consisting of alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, aryl, aryl, cycloalkyl having 3 to 6 ring members, cycloalkenyl having 4 to 6 ring members, cycloalkylalkyl having 3 to 6 ring members, cycloalkenylalkyl having 4 to 6 ring members, with the proviso that each of the foregoing R₃ substituents has up to 8 carbon atoms, trifluoromethyl, nitro, amino, hydroxyl, halogen, aminocarbonyl, cyano, cyanoalkyl having from 2 to 4 carbon atoms, aminocarbonylalkyl having 2 to 4 carbon atoms, aryl, alkaryl, piperazinyl, and methyl-piperazinyl;
(c) X₁ and X₂ are the same or different sterically compatible substituents which are selected from the group consisting of. hydrogen, alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, aryl, cycloalkyl having 3 to 6 ring members, cycloalkenyl having 4 to 6 ring members, cycloalkylalkyl having 3 to 6 ring members, cycloalkenylalkyl having 4 to 6 ring members, with the additional proviso that each of the foregoing X₁ and X₂ substituents has up to 8 carbon atoms, trifluoromethyl, nitro, amino, hydroxyl, halogen, aminocarbonyl, cyano, cyanoalkyl having from 2 to 4 carbon atoms, aminocarbonylalkyl having 2 to 4 carbon atoms; and
(d) X₃ is selected from the group consisting of. a methylene; —C(HR₄)— where R₄ is selected from the group of substituents consisting of alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, aryl, cycloalkyl having 3 to 6 ring members, cycloalkenyl having 4 to 6 ring members, cycloalkylalkyl having 3 to 6 ring members, cycloalkenylalkyl having 4 to 6 ring members, with the additional proviso that each of the foregoing R₄ substituents has up to 8 carbon atoms, trifluoromethyl, nitro, amino, hydroxyl, halogen, aminocarbonyl, cyano, cyanoalkyl having from 2 to 4 carbon atoms, and aminocarbonylalkyl having 2 to 4 carbon atoms; amino; —N(R₅)— where R₅ is selected from the group of substituents consisting of alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, cycloalkyl having 3 to 6 ring members, cycloalkenyl having 4 to 6 ring members, cycloalkylalkyl having 3 to 6 ring members, cycloalkenylalkyl having 4 to 6 ring members, with the additional proviso that each of the foregoing R₅ substituents has up to 8 carbon atoms, trifluoromethyl, nitro, amino, hydroxyl, halogen, aminocarbonyl, cyano, cyanoalkyl having from 2 to 4 carbon atoms, and aminocarbonylalkyl having 2 to 4 carbon atoms; sulfur; phosphorus; and oxygen group; pharmaceutically acceptable salts thereof, enantiomers thereof, or metabolites thereof. The R₁ and R₂ substituents can be on positions 3, 4, 5, or 6 of the ring.

The invention includes compounds having the structural formula 1 and the acid addition salts thereof. For medical use, the pharmaceutically acceptable acid addition salts are preferred. The pharmaceutically acceptable acid addition salts are those salts in which the anion does not contribute significantly to the toxicity or pharmacological activity of the salt, and as such, they are the pharmacological equivalents of the bases having the foregoing structural formulas. In some instances, the salts have physical properties which make them more desirable for pharmaceutical formulation purposes such as solubility, lack of hygroscopicity, compressibility with respect to tablet formation and compatibility with other ingredients with which the substances may be used for pharmaceutical purposes.

The acid addition salts are made by reaction of a base of the structural formula 1 with the acid, preferably by contact in solution. They also are made by metathesis or treatment with an anion exchange resin whereby the anion of one salt of the substance is replaced by another anion under conditions which allows for separation of the undesired species such as by precipitation from solution or extraction into a solvent or elution from or retention on an anion exchange resin. Pharmaceutically acceptable acids for the purposes of salt formation include hydrochloric, hydrobromic, hydroiodic, citric, acetic, benzoic, phosphoric, nitric, mucic, isethionic, methanesulfonic, p-toluenesulfonic, glucosaccharic, palmitic, heptanoic, oxalic, cyclamic, succinic, malic, fumaric, mandelic, malonic, and others.

The compounds of the present invention shown by the structural formula 1 contain an asymmetric carbon atom in the cyclopyrrolone ring and occur as optically active isomers as well as racemic mixtures thereof. The present invention is intended to include each of the optically pure, optically active and racemic forms. Some of the substances of the present invention contain an asymmetric carbon atom in the X₁, X₂, R₁, R₂, or R₃ substituents in formula I, and diastereoisomeric pairs of racemates exist. These forms are also contemplated and included in the methods of the present invention.

Resolution of racemic mixtures to provide the optically active isomers of the foregoing compounds is carried out, for example, by forming a salt with an optically active acid many of which are known to those skilled in the art such as optically active tartaric, mandelic, cholic, O,O-di-p-toluoyl tartaric, and O,O-dibenzoyl tartaric acids, or other acids conventionally employed for this purpose. Separation of optical isomers can be accomplished as disclosed in U.S. Pat. No. 4,960,779 which is incorporated herein by reference.

The invention relates in particular to transdermal administration of pagaclone:

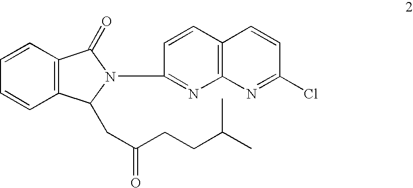

Another preferred embodiment is transdermal administration of the pagaclone metabolite of formula 8, described in U.S. Pat. No. 5,676,831; incorporated by reference in its entirety.

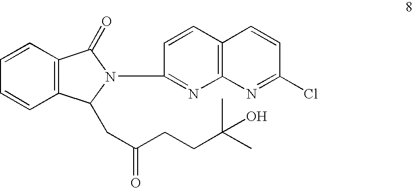

The compounds of formula 1 can be prepared by the action of a ketone of formula:
CH₃—CO—R₃ (3)
in which R₃ is as defined above, with a 3-hydroxyisoindolinone of formula 4: 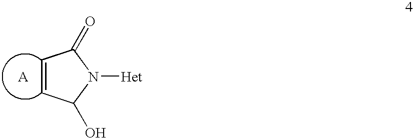
in which A is the ring formed by X₁ and X₂ above and Het is the heterocyclce defined in formula 1.

The reaction is generally performed in a dipolar aprotic solvent such as dimethylformamide or N-methyl-2-pyrrolidone, in the presence of a base such as an alkali metal hydride, e.g. sodium hydride, at a temperature of between −10 and +60 degreed Centigrade.

The compounds of general formula (4) may be prepared by application or adaptation of the methods described in Belgian Pat. Nos. 793,851, 835,325 and 815,019.

According to a further feature of the invention, the products of general formula 1 are prepared by the action of a beta-keto ester of formula 5:
R₄OOCCH₂COR₃ (5)
which R₄ denotes alkyl and R₃ is as defined above, on a compound of formula 6: 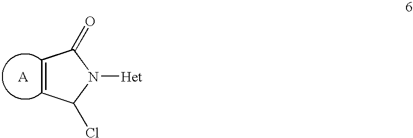
in which Het is defined above.

The condensation of the keto-ester of formula (5) with the compound of formula (6) is generally performed in an organic solvent in the presence of a base, e.g. in dimethylformamide or tetrahydrofuran in the presence of an alkali metal hydride such as sodium hydride, at a temperature of between 0 and 60 degrees Centigrade and preferably between 20 and 60 degrees C.

The subsequent dealkyloxycarbonylation may be performed by any method known to those skilled in the art, in particular by alkaline saponification followed by an acidification and heating to a temperature of between 100 and 200 C., by acid hydrolysis and attendant decarboxylation at a temperature of between 100 and 200 C., or alternatively by heating in dimethyl sulphoxide in the presence of an alkali metal halide, e.g. lithium chloride, at a temperature of between 150 and 180 C.

The products of general formula (6) may be prepared by chlorination of a product of general formula (4). The reaction is generally performed in the presence of a chlorinating agent such as sulphinyl chloride or phosphorus oxychloride, in the presence of catalytic amounts of dimethylformamide, at a temperature between 20 C. and the refluxing temperature of the reaction mixture, or any other agent known to those versed in the art which enable a hydroxy radical to be converted to a chloro radical without affecting the remainder of the molecule.

The products of general formula 1 may be purified by the usual known methods, e.g. by crystallization, chromatography or successive extractions in acidic and basic medium.

The products of general formula 1 may be converted to an addition salt with acids, by the action of an acid in water or in an organic solvent such as an alcohol, a ketone, an ether or a chlorinated solvent. The salt formed precipitates, where appropriate after concentration of its solution; it is separated by filtration or after settling has occurred.

The products of general formula 1 possess especially advantageous pharmacological properties, and have an anxiolytic, hypnotic, anticonvulsant, antiepileptic and muscle relaxant activity. Thus, they show appreciable affinity in vitro for benzodiazepine receptor sites at concentrations between 0.4 and 200 nM according to the technique described by J. C. BLANCHARD and L. JULOU, J. of Neurochemistry, 40, 601 (1983) modelled on the work of SQUIRES and BRAESTRUP, Nature, 266, 732 (1977).

In animals (mice), they have been shown to be active, at doses which are generally between 0.5 and 200 mg/kg orally, with respect to pentetrazole-induced convulsions according to a technique closely allied to that of EVERETT and RICHARDS, J. Pharmacol., 81, 402 (1944).

The products of general formula 1 and their salts possess, in addition, low toxicity. Their oral LD.sub.50 is generally greater than 300 mg/kg in mice.

For medicinal use, the products of general formula (I) may be employed as they are or in the state of pharmaceutically acceptable salts, i.e. salts which are non-toxic at the doses at which they are used.

The target plasma level for pagaclone is between 0.09 ng/ml to 2.5 ng/ml at Cmax. In addition, one would like the active metabolite levels between 5 to 20 ng/ml at Cmax. The amount of pagaclone in a transdemal patch should be from 1 mcg to 5 mg; preferably 50 mcg to 1.5 mg.

As examples of pharmaceutically acceptable salts, there may be mentioned the addition salts with inorganic acids, such as hydrochlorides, sulphates, nitrates and phosphates, or with organic acids, such as acetates, propionates, succinates, benzoates, fumarates, maleates, methanesulphonates, isethionates, theophyllineacetates, salicylates, phenolphthalinates and methylenebis(.beta.-oxynaphthoates), or substitution derivatives of these compounds.

Of special value are the products of general formula (1) in which A forms with the pyrrole ring an isoindoline or 6,7-dihydro-5H-pyrrolo[3,4-b]pyrazine ring-system, Het denotes a 1,8-naphthyridin-2-yl or 2-quinolyl radical which is unsubstituted or substituted by halogen, (1 to 4 C) alkyl, (1 to 4 C) alkoxy or (1 to 4 C) alkylthio, and R₃ denotes straight- or branched-chain alkenyl of 3 to 6 carbon atoms or an alkyl radical which is unsubstituted or substituted by hydroxy, alkyloxy, cycloalkly of 3 to 6 carbon atoms, dialkylamino, dialkylcarbamoyl or phenyl, or R₃ denotes 4-piperidyl or cyclohexyl, the alkyl radicals being straight- or branched-chain radicals and containing 1 to 10 carbon atoms each, and said piperidyl radical may be substituted at the 1-position by alkyl.

The following products are of very special value:
2-(7-chloro-1,8-naphthyridin-2-yl)-3-(5-methyl-2-oxohexyl)-1-isoindolinone
2-(7-methoxy-1,8-naphthyridin-2-yl)-3-(2-oxohexyl)-1-isoindolinone
2-(7-methoxy-1,8-naphthyridin-2-yl)-3-(5-methyl-2-oxohexyl)-1-isoindolinone
2-(7-methoxy-1,8-naphthyridin-2-yl)-3-(6-methyl-2-oxo-5-heptenyl)-1-isoindolinone
2-(7-methoxy-1,8-naphthyridin-2-yl)-3-(6-methyl-2-oxoheptyl)-1-isoindolinone
3-(3-cyclohexyl-2-oxopropyl)-2-(7-methoxy-1,8-naphthyridin-2-yl)-1-isoindolinone
2-(7-chloro-1,8-naphthyridin-2-yl)-3-(3-isopropoxy-2-oxopropyl)-1-isoindolinone
2-(7-chloro-2-quinolyl)-3-(5-methyl-2-oxohexyl)-1-isoindolinone
2-(7-fluoro-1,8-naphthyridin-2-yl)-3-(5-methyl-2-oxohexyl)-1-isoindolinone.

For adequate skin penetration of most drugs, a chemical permeation enhancer is necessary. As used herein, the term “enhancer” is meant to encompass any enhancer or combination of enhancers that increases the flux of a substance across a mammalian stratum corneum. The enhancer that results in the highest skin flux is often specific to a particular drug and what works for one drug may not work for another. Furthermore, the precise concentration of enhancer and the particular combination of enhancers must be tailored to each drug to achieve the maximum skin flux. There are numerous possible permeation enhancers that can be used and they are typically categorized into two groups, solvent-type enhancers and plasticizing-type enhancers.

Plasticizer-type enhancers refers to fatty acids, fatty acid esters, fatty alcohols and similar hydrophobic compounds that are capable of increasing the permeability of drugs to the stratum corneum. Without limiting the scope of the present invention, the following is proposed as the mechanism of action of the plasticizer-type enhancers. It is believed that the function of the plasticizer-type enhancers is to migrate into the upper stratum corneum layers of the skin and disrupt the lipids which occupy the extracellular spaces of the stratum corneum. The stratum corneum layer, although only 25-50 microns thick, is the principal barrier to transdermal permeation. The plasticizer-type enhancers that migrate into the skin serve to increase the mobility and diffusion of the drug into the skin.

Plasticizer-type enhancers generally will have a molecular weight of greater than 150 but less than 1000. In addition, the plasticizer-type enhancers should also be relatively water insoluble or they will leach into the subcutaneous tissue layers below the stratum corneum. Thus, plasticizer-type enhancers with water solubility of less than 0.5 wt % are preferred, and more preferably 0.2 wt % or less.

Enhancers may also be classified according to their Hildebrand solubility parameters. The Hildebrand solubility parameter measures the cohesive forces and sum of all intermolecular attractive forces related to the extent of mutual solubility of many chemical species. See, eg. the CRC Handbook of Solubility Parameters and other Cohesion Parameters, CRC Press, Inc., Boca Raton, Fla. (1985). Relative hydrophilicityincreases with the value of the Hildebrand solubility parameter (.sigma.). For example, the skin has a .sigma. value of 10, while water has a .sigma. value of 23.4. This implies that enhancers with solubility parameters of <10 will intervene with the lipid component of the skin, but those with solubility parameters of >10 will selectively partition into the polar components of the skin. Generally, plasticizer type enhancers have a .sigma. value of between about 5 and 10.

A preferred group of plasticizer-type enhancers includes lower alkyl and alkoxy esters of pharmaceutically acceptable fatty acids, fatty acid esters, fatty alcohols, and similar hydrophobic compounds. As used herein, the term ‘lower alkyl and lower alkoxy’ refers to alkyl and alkoxy groups having up to and including 7 carbon atoms and preferably, up to and including 4 carbon atoms. Some examples of alkyl groups include methyl, ethyl, propyl, isopropyl, n-butyl, tertiary butyl, pentyl, isopentyl, neopentyl, hexyl, isohexyl and heptyl. Some examples of alkoxy groups include the oxides corresponding to the above alkyl groups. Examples of suitable fatty acid esters include saturated or unsaturated fatty acid esters, including isopropyl myristate, isopropyl palmitate, and the methyl and ethyl esters of oleic and lauric acid. Suitable fatty alcohols include stearyl alcohol and oleyl alcohol. Examples of suitable fatty acids include saturated and unsaturated fatty acids, including oleic acid, lauric acid, myristic acid, palmitic acid, stearic acid, linoleic acid, and palmitoleic acid. In addition, many other compounds can also serve as plasticizer-type enhancers, such as diethyl hexyl phthalate, octyldocecyl myristate, isostearyl isostearate, caprylic/capric triglycerides including polyethylene glycol esters of caprylic/capric acids, propylene glycol laurate (Lauroglycol), Miglyol (propylene glycol diester caproic, caprylic, capric, lauric acid), Lexol PG-865 (propylene glycol diester decanoic, octanoic acid), propylene glycol myristate (mirpyl), corn oil polyethylene glycol—6 esters (Labrafil M2124CS), polyethylene glycol—8 caprylic capric glycerides (Labrasol), caprylic/capric triglycerides (Labrafac Lipophile WL 1349), caprylic/capric triglyceride polyethylene glycol—4 esters (Labrafac Hydro WL11219 available from Gattefosse, Westwood N.J.), glyceryl oleate, hexamethyldisiloxane, m.backslash.dimethicone, cyclomethicone, squalene, mineral oil, macrocyclic ketones/lactones, plant extracts such as Chrodarom Calendula O or Chrodarom Chamomile O, and various oils including wintergreen, jojoba oil, or eucalyptol (cineole).

A preferred plasticizer-type enhancer for use with pagaclone and its metabolite includes caprylic/capric acids triglyceride PEG-4 esters, available as Labrafac Hydro WL 1219, (Gattefosse, Westwood, N.J.) which contains a mixture of saturated polyglycolyzed glycerides consisting of glycerides and polyethylene glycol esters of caprylic and capric acids.

As used herein, “solvent-type enhancer” generally refers to relatively hydrophilic compounds having molecular weights of less than about 200 that are capable of increasing the permeability of drugs to the stratum corneum. Solvent-type enhancers typically exhibit solubility parameters between about 10 and 24, and preferably between about 10 and 18. Solvent-type enhancers are often better enhancers because they generally provide higher flux rates for a given permeant than plasticizer-type enhancers. Typically, the solvent type enhancers will comprise a pharmaceutically-acceptable lower alkyl alcohol, aryl alcohol, or polyol, for example, ethanol, propanol, butanol, benzyl alcohol, glycerin, or propylene glycol. In some embodiments, the solvent-type enhancer is a 2-pyrrolidone or alkyl derivative thereof, such as N-methyl-2-pyrrolidone, 3-hydroxy-N-methyl-2-pyrrolidone, and pyroglutamic acid esters.

Other embodiments may employ an alkyl ether, such as ethylene, polyethylene or propylene glycol ether, as the solvent type enhancer. Preferred examples of ethylene glycol ethers include, but are not limited to, ethylene glycol monoalkyl ethers, such as ethylene glycol monomethyl ether (also known as methyl cellosolve), ethylene glycol dialkyl ethers, such as ethylene glycol dimethyl ether (also known as dimethyl cellosolve), and ethylene glycol monoalkyl ether esters, such as ethylene glycol monoethyl ether acetate (also known as cellosolve acetate). Preferred examples of polyethylene glycol ethers include, but are not limited to, diethylene glycol monoalkyl ethers, such as diethylene glycol monobutyl ether (also known as butyl ethyl Cellosolve or butyl carbitol), diethylene glycol dialkyl ethers; and diethylene glycol monoalkyl ether esters, such as diethylene glycol monoethyl ether acetate (also known as Carbitol acetate), and transcutol (diethylene glycol monoethyl ether).

Preferred solvent type enhancers have a molecular weight of less than about 150. They are also relatively hydrophilic, generally being greater than 2 wt % soluble in water, and are preferably greater than 10 wt % soluble in water. Most preferred solvent type enhancers are completely water miscible. One of skill in the art would appreciate that the solvent type enhancers may be used alone or in combination.

While solvent type enhancers may be useful in delivering large amounts of therapeutic agents through the skin, when used alone, larger amounts of the solvent enhancer must often be applied continuously to achieve a prolonged therapeutic effect, to the extent that they themselves are permeable through the skin. As such, skin irritation may occur.

However, when one or more plasticizer-type enhancers is used in combination with one or more solvent-type enhancers, drugs can be delivered through the stratum corneum at therapeutically effective levels. Such an enhancer mixture achieves high delivery rates of the drug with relatively dilute solutions of the solvent-type enhancer. This tends to eliminate the irritation that occurs when solvent-type enhancers are used alone at high concentrations.

When used with plasticizer-type enhancers, the function of the solvent type enhancer is to rapidly diffuse into the stratum corneum layer of the skin, making it possible for the larger, less mobile plasticizer type enhancers to enter the stratum corneum layer. The small size and hydrophilic nature of the solvent type enhancers makes them very effective in this role.

Patch Configurations

The design of the transdermal patch for use according to the invention involves several factors including the permeability of drug to the skin, the dose of the drug required for the desired therapeutic application, the enhancers used to promote delivery of the drug, and the flux rate of drug and enhancer formulations required to achieve a therapeutic effect. Each active compound has unique physical and chemical properties that influence the design of the patch for effective delivery of the active compound to the patient.

For delivery of the compounds of the present invention, the optimal patch configuration and enhancer combination and concentration is assessed experimentally in vitro using two common methods, skin permeation testing and dissolution release tests. In the dissolution release test, the transdermal device is placed in a volume of receiving fluid sufficient to maintain sink conditions, which is stirred continuously and is maintained at a constant temperature. The cumulative amount of drug or enhancer released into the receiving medium is measured as a function of time. This test allows for confirmation of drug release from the transdermal device as well as a gross discrimination in drug release as a function of transdermal device components and formulation composition.

However, the majority of screening is provided via in vitro skin tests. These tests provide a more realistic picture of the device performance in vivo. In these tests, the test solution or the transdermal device is placed in contact with the skin which is in contact with a receiving phase, usually saline. The amount of drug permeating throughout the skin is determined by monitoring the drug concentration in the receiving phase as a function of time. See, for example, Ritschel and Barkhaus (1987) Meth. and Find. Exptl. Clin. Pharmacol. 9:673-676. These tests are more costly and time consuming than dissolution release tests yet better reflect the actual in vivo performance of the transdermal device. Enhancer selection and composition may be optimized by empirical testing.

Simple Adhesive Matrix Patches

In one embodiment of the invention, the transdermal patch for the delivery of Compound 1 is a simple adhesive patch, which comprises an impermeable backing layer, a release liner, and a drug/adhesive containing matrix.

The impermeable backing layer defines the top of the drug delivery device, i.e., the side furthest away from the skin when the device is in use. The backing forms an occlusive layer that prevents the loss of drug and/or enhancers to the environment and protects the patch from contamination from the environment. The backing layer may be opaque so as to protect the drug from light.

The backing layer can be made from standard commercially available films for medical use, such as those supplied by 3M Corporation, St. Paul, Minn.; Dow Chemical, Midland, Mich.; or AF Packaging, Winston-Salem, N.C. Suitable materials which can be used to form the backing layer include films or sheets of polyolefin, polyester, polyurethane, polyvinyl alcohol, polyvinylidene, polyamide, ethylene-vinylacetate copolymer, ethylene-ethylacrylate copolymer, and the like, metal-vapor deposited films or sheets thereof, rubber sheets or films, expanded synthetic resin sheets or films, unwoven fabrics, fabrics, knitted fabrics, paper, and foils. These materials can be used individually or as laminates. These films can be pigmented or metalized.

Preferred backing layers include Scotchpak.RTM. 1006 and 1009, skin-colored aluminized polyester films of approximately 70-80 mu.m in thickness, and 3M-1012, a transparent polyester film laminate, all of which are available from 3M Corporation.

In some aspects of the invention, the patch may include a peel strip or release liner to cover the surface of the pressure-sensitive adhesive during storage, and prevent evaporative loss of the drug or enhancer(s). The release liner may be formed with dimples for decreasing contacting surface with the adhesive layer, and it may also be formed with a pull-tab for making it easier for removing it from the device.

The peel strip may be made from any impermeable film, such as is specified for the backing layer. Additionally it may be made from metal foil, Mylar™ polyethylene terephthalate, or any material normally used for this purpose in the art that is compatible with the drug and the chosen adhesive. Examples of suitable compositions for the release liner include siliconized polyester, poly (1,1-dihydroperfluoroctylmethacrylate), fumed silica in silicone rubber, end-capped siliconized polyethylene terephthalate, polytetrafluoroethylene, cellophane, a film of polyvinyl chloride having titanium dioxide dispersed therein, and the like. Preferred release liners include a silicon coated Release Technology 381B and fluoropolymer coated polyester films, such as 3M Scotchpak.RTM. 1022 film.

In the simple adhesive matrix patch, the drug depot layer is comprised of the drug and an adhesive, the layer attaching directly to the skin of the patient after the peel strip or release liner is removed. In the preferred embodiments of the invention, the drug depot layer also comprises one or more enhancers.

Generally, the selection of the adhesive is important to the proper functioning of the transdermal delivery device. This is particularly true if a plasticizer-type enhancer is placed in the adhesive layer. Specifically, the adhesive layer must retain its functioning properties in the presence of the plasticizer-type and solvent-type enhancers, as well as upon exposure to the alpha blocker. However, often enhancers or other formulation ingredients can compromise the physicochemical and functional properties of an adhesive. Significant loss of cohesive strength can result in undesirable effects such as an increase in tack, cold flow beyond the edge of the patch, transfer of the adhesive to the protective release liner during removal, or adhesive residue left on the skin following removal of the patch. Alternatively, in some cases, the patch loses adhesion altogether and falls off. The loss of tack and other adhesion properties generally dictates and limits the amount and type of enhancers that can be loaded into the adhesive matrix type patches. In addition, as the structural integrity of the dosage unit is lost, the delivery rate of the drug is diminished and/or becomes variable and unstable.

For example, silicon adhesives are commonly used in transdermal delivery devices; however, they are capable of a maximum compatible loading of only about 1% isopropyl myristate. See Pfister et al. (1990) Pharm. Tech. Int. 55-59, Pfister and Hsieh (1991) Pharm. Tech. Int. 3:38-32, and Pfister and Hsieh (1991) Pharm. Tech. Int. 3:32-36. Since the adhesive layer of the devices described herein contain a high level of plasticizer-type enhancer, typically from about 5 to about 50 wt % of a plasticizer-type enhancer, based on the adhesive layer, and preferably from about 10 to about 40 wt %, and more preferably from about 25 to about 35 wt %, the proper selection of the adhesive is important for the performance of these transdermal delivery devices.

Preferably, the adhesives employed in the transdermal delivery systems described herein will have shear values greater than 2, with the higher values being preferred; plasticity values between about 1 and 4, and preferably greater than 1.5, with the higher values being preferred; tack values between about 50 and 1000, with the lower values being preferred; a dynamic loss modulus between about 10⁻⁵ and 10⁻⁷ at frequencies of 10⁻² rad/sec and between 10⁻⁵ and 10⁻⁷ at frequencies of 10² rad/sec; and a dynamic storage modulus between about 10⁻⁵ and 10⁻⁷ at frequencies of 10⁻² rad/sec and between 10⁻⁵ and 10⁻⁷ at frequencies of 102 rad/sec. See Chang (1991) J. Adhesion 34:189-200 and European Patent Publication No. 524,776.

Cross-linked acrylate-based adhesives, such as those available from Avery Chemical Division, Mill Hall, Pa. and National Starch and Chemical Company, Bridgewater, N.J., are able to withstand relatively high loading of enhancers, both solvent-type and plasticizer-type, while still maintaining these performance parameters. These adhesives generally contain about 1 to about 5 wt % of acrylic acid, about 5 to about 20 wt % of a C₄ to a C₁₋₂ alkyl acrylate or alkyl methacrylate. The adhesives also can contain about 0.1 to about 5 wt % of a cross-linking monomer.

Preferred adhesive for delivery of Compound 1 of the present invention are acrylate-based adhesives. Acrylate-based adhesives for use according to the present invention include Avery 2533 and Avery-460HPX adhesives (available from Avery Chemical Division (U.S.), Mill Hall, Pa.), Durotak.RTM. 87-2516, 87-2852, and 87-2287 (available from National Starch, Bridgewater, N.J.), and Monsanto's Gelva GE 1753. The acrylate adhesives Avery 2533 and Avery 460-HPX are similar acrylate adhesives with varying levels of cross linking. The Durotak.RTM. 87-2516, 87-2852 and 87-2287 are acrylic solution pressure sensitive adhesives, with the 2516 and 2852 being cross-linking and the 2287 being non-crosslinking.

Monolithic Matrix Patches

A monolithic matrix patch is an alternative embodiment of the transdermal patch of the invention. The patch comprises an impermeable backing layer, a release liner, a monolithic matrix layer comprising a polymer matrix in which drug is dispersed, and a peripheral adhesive layer. In some embodiments, the patch may also include an optional porous membrane layer. In yet other embodiments, the patch may have an adhesive layer that is co-extensive with the skin facing surface of the patch. The monolithic matrix layer comprises the drug, and one or more enhancers dispersed in a polymeric matrix.

The monolithic matrix layer may also comprise additional components such as diluents, stabilizers, vehicles, biocides, antioxidants, anti-irritants and the like. A preferred embodiment of the monolithic matrix patch is a monolithic matrix patch with a peripheral adhesive annular ring and a drug depot having a hydrogel matrix or a foam matrix.

Reservoir Type Patches

A further embodiment of the invention is the reservoir type patch which allows a higher loading level of active material, and usually, a higher loading level of enhancer. Such a patch is comprised of an impermeable backing layer which is sealed at its periphery to an inert membrane, thereby defining between these two layers a drug depot. An adhesive layer is affixed to the skin facing side of the patch. The patch also comprises a release liner. The drug depot contains the drug, and optionally one or more enhancers or gelling components. Thus, in the reservoir type patches, a membrane separates the drug reservoir from the adhesive layer. In some embodiments the membrane is a non-rate controlling membrane. According to the present invention, a non-rate controlling membrane is one in which the rate of permeation of the enhancer(s) and drug through the membrane is greater than their permeation rate through the skin or any other portion of the device (typically two to five times greater or more). Thus, a non-rate controlling membrane is extremely permeable to the enhancer(s) and the drug contained in the reservoir.

In other embodiments, the membrane may be a rate-controlling membrane. As used herein, a rate-controlling membrane is one in which the rate of permeation of the enhancer(s) and the drug through the membrane is less than or equal to their permeation rate through the skin or any other portion of the device. Rate-controlling membranes are described, for example, in U.S. Pat. Nos. 4,460,372 and 4,379,454.

The membrane may comprise a microporous or porous material. Microporous membranes have a distinct pore structure with pores ranging in diameter from approximately 0.08 to 0.5 microns, preferably from about 0.1 and 0.4 microns, and more preferably from about 0.2 and 0.4 microns. Examples of suitable microporous membranes include polyethylene and polypropylene films, nylon, and nitrocellulose film. A preferred membrane material is Cotran TM. 9710, which is a polyethylene membrane, 50 mu.m in thickness, with a void volume of greater than 10%, available from 3M Corporation. Other embodiments of the present invention will utilize other microporous polyethylene membranes, such as Celgard K-256, available from Hoechst-Celanese, Charlotte, N.C. Porous membranes have pores greater than about 3 microns in diameter. Such materials are available as woven and non-woven fabrics, such as non-woven polyester #9770 from Dexter Corp. (Windsor Locks, Conn.). These materials can also be fabricated from nylon, polyethylene, polyolefins and the like.

In the reservoir type patches, the membrane and the backing layer are sealed at their peripheral edges to form the drug reservoir. This seal should be substantially fluid-tight to prevent drug leakage from the reservoir through the seal between the backing layer and the membrane. As used herein, the term “peripheral edges” of the membrane and backing layers refer to the areas that are sealed together to define the drug reservoir. Therefore, extraneous membrane and backing layer material may extend outwardly from the drug reservoir and peripheral edge.

The drug reservoir contains a solution, suspension, or gel of the drug and the permeation enhancers, as well as diluents, such as water, and vehicles or other additives. The drug can be dispersed in the solution, suspension, or gel in either a dissolved or undissolved state.

A gelling agent may be incorporated into the reservoir or monolithic matrix to increase the viscosity and rheological characteristics of the drug and enhancers. The agent also serves to prevent settling of the dispersed drug during storage. Typically, a viscosity range of about 100 to 100,000 centipoise for the combination of materials forming the reservoir is necessary in order to produce the drug delivery device using form-filling technology, as described in greater detail below.

The gelling agent comprises a pharmaceutically-acceptable material that is capable of increasing viscosity of the reservoir solution. Typically, the drug delivery devices described herein will employ cellulosic materials as the gelling agent. Examples of suitable cellulosic materials include cellulose, cellulose derivatives, alkylcellulose, hydroxy-(lower alkyl) cellulose derivatives where the alkyl group contains one to six carbons, carboxyalkylcellulose and the like. Other gelling agents include PVP, CMC, Klucel, alginates, kaolinate, bentonite, or montmorillonite, other clay fillers, stearates, silicon dioxide particles, carboxy polymethylene, ethylene maleic anhydride, polyacrylamide, and poly (methyl vinyl ether maleic anhydride.)

A preferred embodiment of the present invention utilizes a hydroxy-(lower alkyl) cellulose as the gelling agent. Typically, hydroxypropylcellulose will be employed in an amount from about 0.1 to about 20 wt %, based on the reservoir fill solution, and preferably from about 0.5 to about 10 wt %. In the example described below, the gelling agent is present in about 2 wt

The reservoir or matrix layer also may include diluents, stabilizers, vehicles, biocides, antioxidants, anti-irritants and the like. For example, water is frequently utilized as a diluent in the reservoir type patches. Typically water will be present in the reservoir in an amount not greater than about 50 wt %, based on the reservoir fill solution; preferably, not greater than 40 wt %. Other diluents which will frequently find use in the drug delivery devices described herein include glycerine and propylene glycol.

A pressure-sensitive adhesive layer, is affixed to the membrane opposite to the backing layer. Ideally, the adhesive layer should interact minimally with the drug; it should adhere firmly to the membrane, but removably to the peel strip; it should stick securely to the wearer for extended periods, yet allow the transdermal delivery device to be removed with minimum discomfort; and it should not give rise to undue skin irritation, allergic reactions or other dermatological problems. These properties must be maintained from the time of patch manufacture, throughout storage, and up to and throughout the time of application.

An alternative embodiment of the reservoir patch has a peripheral adhesive, wherein the area of the adhesive layer is not co-extensive with the active releasing area of the patch, but rather forms an annular ring around the active releasing area of the patch. The delivery of the drug thus is not primarily through the adhesive layer of the patch, although some lateral diffusion may occur within the patch, resulting in delivery of active substance through the adhesive at the periphery of the patch. The shape of the peripheral adhesive region will vary with the shape of the patch, but will generally comprise the outer perimeter of the patch, in order that an adequate adhesive seal is maintained between the skin and the patch to prevent the patch from falling off. The percentage of the patch that comprises the peripheral adhesive portion depends on the type of adhesive, the type of backing layer, the length of time the patch will be worn, and the weight and loading of drug in the patch. Such determinations will be apparent to the skilled artisan.

Prior to use, the patches typically are stored in laminate foil pouches, both to prevent contamination and to avoid drug and/or enhancer(s) loss. Such pouches are standard in the industry.

The patch may be assembled by any of the techniques known in the art for producing transdermal patches. The patches may be of various shapes, but the round shape is preferred as it contains no corners and thus is less easily detached from the skin.

The dosage units produced may have various sizes. A total surface area in the range of 1 to 200 cm² is contemplated, with 5-30 cm² being preferred. Depending on the type of patch, the active drug delivery surface area may be somewhat less than the total surface area, for example, in the peripheral adhesive type reservoir patch. Generally the active drug delivery surface area is in the range of 1-190 cm², with 5-30 cm² being preferred.

The percentage by weight of the drug in the solution, hydrogel or matrix may be varied according to the desired loading of the finished patch. The drug content of the finished matrix can vary widely, from about 0.1 to about 70 wt %. Typically, the transdermal patch is applied such that a dose is an amount from 0.1 to 200 mg per day, depending on the therapeutic application.

Generally, therapeutic dosages for compounds of formula 1 for are between 0.001 and 20 mg/kg/of body weight per day, with preferred dosages being between 0.05 and 1 mg/kg of body weight per day, and most preferred dosages being at least approximately 0.3 mg/kg of body weight per day.

The dosage may, however, be more or less depending on factors including therapeutic application, the age of the patient, and the patient's tolerance to the drug, if any. Such dosage changes may be determined by the physician in each case.

Phospholipids that can be used in the composition of this invention include phosphatidylcholine, phosphatidylethanolamine, and phosphatidylserine and mixtures thereof. The fatty acid groups in the phosphatidyl moieties of these phospholipids can be saturated, monounsaturated or polyunsaturated groups such as lauroyl, linoleyl, myristoyl, oleoyl, palmitoyl, and stearoyl groups. Soy lecithin, a mixture of phospholipids rich in monounsaturated and polyunsaturated phosphatidylcholines is particularly preferred.

In the composition of the invention, the concentration of dehydroepiandrosterone compound is in the range of 0.1 to 25 grams per 100 grams of composition, preferably 0.2 to 15 grams per 100 grams, and the concentration of phospholipid is in the range of 1 to 90 grams per 100 grams of composition, preferably 2 to 75 grams per 100 milliliters. The relative proportions of phospholipid to dehydroepiandrosterone compound preferably range from 1:1 to 300:1, most preferably from 2:1 to 250:1.

Dehydroepiandrosterone and phospholipid can be combined with little or no other material present into a concentrate suitable for facilitating the subsequent compounding of a variety of formulations presenting the composition of this invention. Surprisingly, even greater concentrations of dehydroepiandrosterone can be achieved in a mixed solvent system combining phospholipid with ethyl alcohol, cetyl alcohol, and a medium chain length triglyceride. Such concentrates can conveniently include 10 to 15 parts by weight of dehydroepiandrosterone and 85 to 90 parts of phospholipid.

Presentations for use of the composition of this invention can, for example, take the form of pastes, gels, and liquids such as solutions, emulsions, creams, and lotions.

In addition to dehydroepiandrosterone compound and phospholipid, the composition of the invention can include a topically acceptable carrier and such adjuvants as are helpful for convenient dispensing and application of the composition by such presentations as pastes, gels, liquid forms such as solutions, emulsions, creams, and lotions, as well as transdermal delivery systems.

Pastes are liquids whose viscosity is enhanced to the point that flow is largely inhibited by the presence of undissolved as well as dissolved solids which can be waxes or finely divided inorganic solids.

Gels are semisolid systems of either containing suspended small inorganic particles (two phase gels) or organic macromolecules interpenetrated by a liquid (single phase gels).

Solutions are single phase liquids substantially free of solid but small amounts of haze or cloudiness can be tolerated.

Emulsions, lotions, and creams are multiphase liquids containing special components known as surfactants that inhibit or delay the separation of the phases. In the composition of this invention, the phospholipid component can function as surfactant. Added surfactants are therefore not necessary but can be included if desired.

Suitable carriers and adjuvants are selected with a view to being safe in prolonged or even indefinite application of the composition, and include:
Solvents such as ethanol, ethyl acetate, glycerine, polyethylene glycols with average molecular weight ranging from 200 to about 1100, and propylene glycol (water miscible); heptane, purified isoparaffinic hydrocarbons boiling in the range from 60 degree. to 300 degree. C. and fractions thereof, canola oil, olive oil, and mineral oil (not miscible with water);
Emollients such as petrolatum, paraffin wax, beeswax, cetyl palmitate, and lanolin;
Emulsifiers and surfactants such as sodium, potassium, and triethanolamine salts of oleic and stearic acids (which can be prepared in situ by including in the formulation suitable sodium, potassium and amine bases along with the desired acids), dioctyl sodium sulfosuccinate, sodium dodecyl sulfate, glycerol monooleate, glycerol monostearate, and ethoxylated sorbitan esters such as Polysorbate 20, Polysorbate 65 and Polysorbate 80;
Finely divided solids such as aluminum hydroxide, bentonite, kaolin, magnesium silicate, silica, titanium dioxide, and zinc oxide;
Thickeners such as agar, carrageenan, food starch, modified starch, gelatin, gum arabic, guar gum, hydroxyethylcellulose, hydroxypropyl methylcellulose, pectin, sodium carboxymethylcellulose and polyacrylic acid adjusted in pH to provide the desired extent of thickening;
Antioxidants and preservatives such as benzalkonium chloride, di-coco-dimethylammonium chloride, dilauryl thiodipropionate, methyl parahydroxybenzoate, propyl parahydroxybenzoate, and tocopherol.

Particularly preferred carriers and adjuvants include medium chain length triglycerides having six to ten carbon atoms in each fatty acid chain, straight chain aliphatic alcohols having twelve to twenty carbon atoms, ethanol, and water. Examples of suitable medium chain length triglycerides include tricaprylin, tricaprin, and a high purity mixed C₈-C₁₀ triglyceride available from Unilever GmbH of Hamburg, Germany, under the trade name HB-307. Examples of suitable straight chain aliphatic alcohols include behenyl alcohol, cetyl alcohol, lauryl alcohol, myristyl alcohol, oleyl alcohol, stearyl alcohol and mixtures thereof.

When formulated for presentation as a solution, the composition of the invention can include volatile carriers such as ethanol and water as well as non-volatile carriers such as medium chain length triglyceride and straight chain aliphatic alcohols having twelve to twenty carbon atoms to supplement or substitute for volatile carriers. Thus a typical solution composition of the invention includes a concentration of dehydroepiandrosterone compound in the range of 0.5 to 15 grams per 100 grams of composition, preferably 2 to 15 grams per 100 grams, a concentration of phospholipid in the range of 5 to 75 grams per 100 grams of composition, preferably 10 to 65 grams per 100 grams, a concentration of volatile carrier in the range of 0 to 90 grams per 100 grams, and a concentration of non-volatile carrier in the range of 0 to 30 grams per 100 grams.

When formulated for presentation as a lotion, the composition of the invention can, if desired, include a finely divided solid and a thickener. Thus a typical lotion composition of the invention includes a concentration of dehydroepiandrosterone compound in the range of 0.2 to 10 grams per 100 grams of composition, preferably 1 to 5 grams per 100 grams, a concentration of phospholipid in the range of 2 to 30 grams per 100 grams of composition, preferably 4 to 25 grams per 100 grams, a concentration of finely divided solid in the range of 0 to 5 grams per 100 grams of composition and a concentration of thickener in the range of 0 to 5 grams per 100 grams of composition.

When formulated for presentation as a cream, the composition of the invention can, if desired, include an emollient and an emulsifier, as well as an antioxidant and/or preservative. Thus a typical cream composition of the invention includes a concentration of dehydroepiandrosterone compound in the range of 0.1 to 10 grams per 100 grams of composition, preferably 0.25 to 5 grams per 100 grams; a concentration of phospholipid in the range of 2 to 75 grams per 100 grams of composition, preferably 3 to 65 grams per 100 grams; a concentration of emollient in the range of 0 to 50 per 100 grams of composition and a concentration of emulsifier in the range of 0 to 25 grams per 100 milliliters of composition.

When formulated for presentation as a paste, the composition of the invention can, if desired, include a thickener and/or finely divided solid in greater concentrations than in a lotion. Thus a typical paste composition of the invention includes a concentration of dehydroepiandrosterone compound in the range of 1 to 10 grams per 100 grams of composition, preferably 2 to 5 grams per 100 grams; a concentration of phospholipid in the range of 2 to 50 grams per 100 grams of composition, preferably 5 to 40 grams per 100 grams; a concentration of finely divided solid in the range of 0 to 15 grams per 100 grams of composition, and a concentration of thickener in the range of 0 to 15 grams per 100 grams of composition, the combined concentration of finely divided solid and thickener being at least 5 grams per 100 grams of composition.

When formulated for presentation as a gel, the composition of the invention can include a gelling agent such as a finely divided solid and/or a thickener in concentrations that produce a loose molecular network inhibiting the free movement of liquid ingredients. Thus a typical gel composition of the invention includes a concentration of dehydroepiandrosterone compound in the range of 0.1 to 10 grams per 100 grams of composition, preferably 0.25 to 5 grams per 100 grams; a concentration of phospholipid in the range of 2 to 50 grams per 100 grams of composition, preferably 3 to 25 grams per 100 milliliters; a concentration of finely divided solid in the range of 0 to 15 grams per 100 grams of composition, and a concentration of thickener in the range of 0 to 15 grams per 100 grams of composition, the combined concentration of finely divided solid and thickener being at least 1 gram per 100 grams of composition.

In a particularly preferred embodiment, the composition of this invention is administered to the recipient by means of a transdermal delivery system or patch. Transdermal delivery is accomplished by exposing a source of the substance to be administered to the recipient's skin for an extended period of time. Typically, the substance is incorporated in a matrix or container from which it is released onto the recipient's skin. The rate of release can be controlled by a membrane placed between the container and the skin, by diffusion directly from the container, or by the skin itself serving as a rate-controlling barrier. Many suitable transdermal delivery systems and containers therefor are known, ranging in complexity from a simple gauze pad impregnated with the substance to be administered and secured to the skin with an adhesive bandage to multilayer and multicomponent structures. All such systems are characterized by the use with the substance to be administered of a shaped article sufficiently flexible to snugly fit to the skin of the recipient and thus serve both as container from which the substance is delivered to the recipient's skin and as barrier to prevent loss or leakage of the substance away from the area of the skin to which the substance is to be delivered. For brevity, such a flexible article is referred to in the instant specification and claims as a reservoir.

Typically, a transdermal delivery system or patch also contains an added substance that assists the penetration of the active ingredient through the skin, usually termed a skin enhancer or penetration enhancer. Many penetration enhancers are known in the art, both water soluble and water insoluble. It has been discovered in accordance with this invention that the phospholipid component of the composition of this invention is outstandingly effective in assisting the penetration of a dehydroepiandrosterone compound through the skin and the establishment of increased serum concentrations of dehydroepiandrosterone sulfate in the recipient.

Accordingly, a transdermal delivery system according to this invention comprises a reservoir, a dehydroepiandrosterone compound as the active ingredient, and a phospholipid compound as penetration enhancer in sufficient concentration to effect increased serum concentration of dehydroepiandrosterone in the recipient. Conventional penetration enhancers are not necessary but can be included if desired.

Compositions according to this invention can be prepared by conventional procedures. To minimize contamination from the growth of microorganisms, sterilized equipment is preferably used. Once blended, the composition can be packaged and stored in any suitable container inert to the contents including aluminum, glass, stainless steel, and solvent resistant plastics including polyamide, polyester, polypropylene, and ABS polymer. Storage is preferably in a cool place away from strong light. Continued sterility can be assured by conventional techniques including aseptic packaging and post-sterilization in the final package by electron beam exposure.

In use, compositions according to this invention are applied to areas of the skin of the recipient in any suitable manner. Thus, a solution or emulsion of the composition can be brushed or painted on desired areas of the recipient's body. A paste, gel, cream, or lotion can be taken on the palm of the hand and rubbed into the recipient's shoulder area, chest, abdominal area, buttocks, or thighs. Transdermal patches can be applied to the upper arm or any suitable less visible area.

Delivery of dehydroepiandrosterone to the recipient's blood stream can be confirmed by analysis of a blood sample taken from the recipient. Increased serum levels of dehydroepiandrosterone sulfate are noted within twenty-four hours and continue to increase to a plateau of at least twice baseline levels and beneficial effects thereof are noted within two weeks.

The invention is now further illustrated by the following examples which are exemplary but not scope limiting.

### EXAMPLE 1

### Preparation of Adhesive Patch

An acrylate adhesive casting solution (Durotak 2516) is prepared containing 10% of a compund of formula 1 and 20% N-methyl-2-pyrrolidone. The casting solution is cast onto a silicone coated release liner to a thickness of 50-200 .mu.m and dried at room temperature for 20 min. and then at 70 dgrees C. for 30 min. A polyester backing (3M 1220) is adhered to the dried adhesive film which is then punched into patches of the desired size.

### EXAMPLE 2

Preparation of an Emulsion Containing Pagaclone and Phosphatidylcholine

A mixture of 23 grams cetyl alcohol and 24 grams petrolatum is warmed to 75 degrees C. to give a clear melt, to which are added 1 gram of pagaclone and 2 grams phosphatidylcholine. Separately, 1 gram sodium lauryl sulfate, 12 grams propylene glycol, 25 milligrams of methyl p-hydroxybenzoate and 15 milligrams of propyl p-hydroxybenzoate are dissolved in 37 grams of warm water, heated to 75 degrees C., and stirred into the melted first mixture. Stirring is continued with cooling until the resulting oil-in-water emulsion sets into a washable ointment containing approximately 1000 milligrams of pagaclone per 100 ml and 2000 milligrams of phosphatidylcholine per 100 ml in accordance with this invention.

1. A transdermal patch for topical administration of a cyclopyrrolone compound, comprising:
a) a backing layer;
b) a drug depot selected from the group consisting of a liquid reservoir and a monolithic matrix; said depot comprising a compound of formula 1, and a permeation enhancer composition; and
c) means for affixing the patch to the skin of the patient;
formula 1: 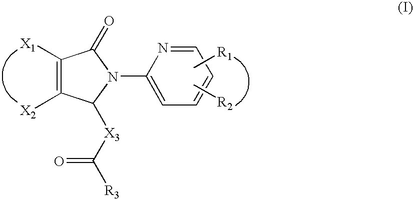
wherein:
(a) R₁ and R₂ are the same or different sterically compatible substituents which are selected from the group consisting o£ hydrogen; alkyl having 1 to 8 carbon atoms; alkyl having 1 to 8 carbon atoms, and having at least one of nitrogen, oxygen, sulfur, or phosphorus; aryl having 1 to 8 carbon atoms; and aryl having 1 to 8 carbon atoms and having at least one nitrogen, oxygen, sulfur, or phosphorus;
(b) R₃ is selected from the group of substituents consisting of alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, aryl, aryl, cycloalkyl having 3 to 6 ring members, cycloalkenyl having 4 to 6 ring members, cycloalkylalkyl having 3 to 6 ring members, cycloalkenylalkyl having 4 to 6 ring members, with the proviso that each of the foregoing R₃ substituents has up to 8 carbon atoms, trifluoromethyl, nitro, amino, hydroxyl, halogen, aminocarbonyl, cyano, cyanoalkyl having from 2 to 4 carbon atoms, aminocarbonylalkyl having 2 to 4 carbon atoms, aryl, alkaryl, piperazinyl, and methyl-piperazinyl;
(c) X₁ and X₂ are the same or different sterically compatible substituents which are selected from the group consisting of hydrogen, alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, aryl, cycloalkyl having 3 to 6 ring members, cycloalkenyl having 4 to 6 ring members, cycloalkylalkyl having 3 to 6 ring members, cycloalkenylalkyl having 4 to 6 ring members, with the additional proviso that each of the foregoing X₁ and X₂ substituents has up to 8 carbon atoms, trifluoromethyl, nitro, amino, hydroxyl, halogen, aminocarbonyl, cyano, cyanoalkyl having from 2 to 4 carbon atoms, aminocarbonylalkyl having 2 to 4 carbon atoms; and
(d) X₃ is selected from the group consisting of a methylene; —C(HR₄)— where R₄ is selected from the group of substituents consisting of alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, aryl, cycloalkyl having 3 to 6 ring members, cycloalkenyl having 4 to 6 ring members, cycloalkylalkyl having 3 to 6 ring members, cycloalkenylalkyl having 4 to 6 ring members, with the additional proviso that each of the foregoing R₄ substituents has up to 8 carbon atoms, trifluoromethyl, nitro, amino, hydroxyl, halogen, aminocarbonyl, cyano, cyanoalkyl having from 2 to 4 carbon atoms, and aminocarbonylalkyl having 2 to 4 carbon atoms; amino; —N(R₅)— where R₅ is selected from the group of substituents consisting of alkyl, alkenyl, alkynyl, alkoxy, alkenoxy, alkynoxy, alkoxyalkyl, alkanoyl, alkenoyl, alkanoyloxy, alkenoyloxy, alkylsulfonyl, alkylsulfinyl, alkylthio, alkanoylamino, alkenoylamino, alkoxycarbonyl, alkenoxycarbonyl, alkoxycarbonylamino, alkoxycarbonylaminoalkyl, cycloalkyl having 3 to 6 ring members, cycloalkenyl having 4 to 6 ring members, cycloalkylalkyl having 3 to 6 ring members, cycloalkenylalkyl having 4 to 6 ring members, with the additional proviso that each of the foregoing R₅ substituents has up to 8 carbon atoms, trifluoromethyl, nitro, amino, hydroxyl, halogen, aminocarbonyl, cyano, cyanoalkyl having from 2 to 4 carbon atoms, and aminocarbonylalkyl having 2 to 4 carbon atoms; sulfur; phosphorus; and oxygen group; pharmaceutically acceptable salts thereof, enantiomers thereof, or metabolites thereof.

2. The transdermal patch of claim 1, containing 1-90 grams of permeation enhancer per 100 grams of composition.

3. A transdermal patch according to claim 1 for achieving an above baseline serum concentration of said compound within two days of beginning administration.

4. A transdermal patch according to claim 1 for maintaining the plasma concentration of a dose of said compound for at least four hours longer than by oral ingestion.

5. A transdermal patch according to claim 1 in which the compound has the structure of formula 7:

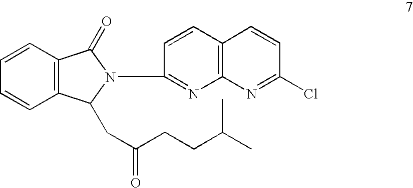

6. A transdermal patch according to claim 1 in which the compound has the structure of formula 8:

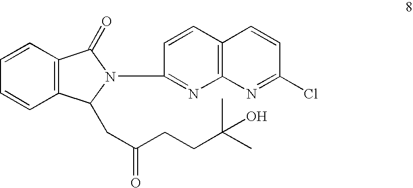

7. A transdermal patch according to claim 1 in which the permeation enhancer is phosphatidylcholine.

8. A transdermal patch according to claim 1 in which the permeation enhancer is phosphatidylethanolamine.

9. A transdermal patch according to claim 1 in which the permeation enhancer is phosphatidylserine.

10. A transdermal patch according to claim 7 in which the permeation enhancer is soy lecithin.

11. A transdermal patch according to claim 1 additionally including at least one topically acceptable carrier selected from the group consisting of medium chain triglycerides having six to ten carbon atoms in each fatty acid chain, straight chain aliphatic alcohols having twelve to twenty carbon atoms, ethanol, and water.

12. A transdermal patch according to claim 11 which is a solution including a concentration of compound 1 in the range of 0.5 to 15 grams per 100 grams of composition, a concentration of phospholipid in the range of 5 to 75 grams per 100 grams of composition, a concentration of volatile carrier in the range of 0 to 90 grams per 100 grams of composition, and a concentration of non-volatile carrier in the range of 0 to 30 grams per 100 grams of composition.

13. A transdermal patch according to claim 1 which is a cream including a concentration of compound 1 in the range of 2 to 75 grams per 100 grams of composition, a concentration of emollient in the range of 0 to 50 grams per 100 grams, and a concentration of emulsifier in the range of 0 to 30 grams per 100 grams.

14. A transdermal patch according to claim 1 in which is a lotion including a concentration of compound 1 in the range of 0.2 to 10 grams per 100 grams of composition, a concentration of phospholipid in the range of 2 to 30 grams per 100 grams of composition, a concentration of finely divided solid in the range of 0 to 5 grams per 100 grams of composition, and a concentration of non-volatile carrier in the range of 0 to 5 grams per 100 grams of composition.

15. A transdermal patch according to claim 1 in which is a gel including a concentration of compound 1 in the range of 0.1 to 10 grams per 100 grams of composition, a concentration of phospholipid in the range of 2 to 50 grams per 100 grams of composition, a concentration of phospholipid in the range of 2 to 50 grams per 100 grams of composition, a concentration of finely divided solid in the range of 0 to 15 grams per 100 grams of composition, and a concentration of thickener in the range of 0 to 15 grams per 100 grams of composition, provided that the combined concentration of finely divided solid and thickener is at least 1 gram per 100 grams of composition.

16. A stable liquid concentrate suitable for preparing a composition according to claim 1, comprising 10 to 15 parts by weight of compound 1, 50 to 80 parts by weight of phospholipid, 10 to 100 parts by weight of ethanol, 2 to 20 parts by weight of medium chain length triglyceride, and 1 to 10 parts by weight of cetyl alcohol.

17. A transdermal patch suitable for a once-a-week administration of pagoclone, an active metabolite thereof, or prodrugs of the foregoing comprising an amount of pagoclone, an active metabolite thereof, or prodrugs of the foregoing contained in a transdermal delivery system for the controlled release of pagoclone, an active metabolite thereof, or prodrugs of the foregoing, such that an average plasma level of pagoclone, an active metabolite thereof, or prodrugs of the foregoing falling in the range of about 0.05 ng/ml to about 25 ng/ml is achieved over a one-week period.

18. The transdermal patch of claim 17 in which said average plasma level is measured at Cmax.

19. The transdermal patch of claim 17 in which an average plasma level of pagoclone falling in the range of about 0.09 ng/ml to about 2.5 ng/ml is achieved over a one-week period.

20. The transdermal patch of claim 17 in which an average plasma level of an active metabolite of pagoclone falling in the range of about 5 ng/ml to about 20 ng/ml is achieved over a one-week period.

21. The transdermal patch of claim 17 which contains an effective amount of pagoclone, an active metabolite thereof, or prodrugs of the foregoing falling in the range of about 7 mcg to about 70 mg.

22. A transdermal patch suitable for a once-a-week administration of pagoclone, an active metabolite thereof, or prodrugs of the foregoing comprising an amount of pagoclone, an active metabolite thereof, or prodrugs of the foregoing contained in a transdermal delivery system for the controlled release of pagoclone, an active metabolite thereof, or prodrugs of the foregoing, such that once-a-week application of said transdermal patch provides steady state blood levels of pagoclone, an active metabolite thereof, or prodrugs of the foregoing, which are substantially bioequivalent to steady state blood levels of pagoclone, an active metabolite thereof, or prodrugs of the foregoing achieved with once daily oral administration of pagoclone, an active metabolite thereof, or prodrugs of the foregoing at an oral daily dosage level falling in the range of about 0.1 mg to about 1 mg.
